(19) United States
(12) Patent Application Publication
(10) Pub. No.: US 2026/0217727 A1
(43) Pub. Date: Jul. 30, 2026

(54) COMPOUNDS DERIVED FROM DIHYDROPIRIDINES, PHARMACEUTICAL COMPOSITIONS COMPRISING SAID COMPOUNDS AND METHOD OF TREATMENT OF MUSCLE DISORDERS COMPRISING ADMINISTRATION OF SAID COMPOSITIONS

(71) Applicant: UNIVERSIDAD DE CHILE, Santiago (CL)

(72) Inventors: Mariana Victoria CASAS ATALA, Santiago (CL); Enrique Zacarías JAIMOVICH PÉREZ, Santiago (CL)

(21) Appl. No.: 19/147,603

(22) PCT Filed: Jan. 13, 2023
(86) PCT No.: PCT/IB2023/050343

§ 371 Date: Jul. 11, 2025

(51) Int. Cl. C07D 491/048; A61K 31/4355; A61P 21/00

(52) U.S. Cl. CPC C07D 491/048; A61K 31/4355; A61P 21/00

(57) ABSTRACT

The invention of the present application relates to compounds derived from tetrahydrofuran [3,4-b]pyridine-3-carboxylate, pharmaceutical compositions comprising said compounds and a method of prevention and treatment of muscle disorders comprising administration of said pharmaceutical compositions for recovery of muscular function in a subject in need thereof.

### FIELD OF THE INVENTION

The invention of the present application relates to compounds derived from tetrahydrofuran [3,4-b]pyridine-3-carboxylate, pharmaceutical compositions comprising said compounds and therapeutic use of said pharmaceutical composition in the recovery of muscular function.

### BACKGROUND OF THE INVENTION

Muscles are organs made up primarily of contractile fibers that help move and are part of how the body works. Each muscle is made up of thousands, or even tens of thousands of small fibers, that contributes to its elasticity and contractility properties. The body has three types of muscles: smooth muscle, cardiac muscle, and skeletal muscle, where these different types of muscles have different functions, any of which are susceptible to many problems or diseases that can cause weakness, pain, or even paralysis.

Some known causes to said problems are: injury or overuse (such as sprains and strains, cramps, or tendonitis); genetics, such as muscular dystrophy; cancer; inflammation, such as myositis; nervous system diseases affecting muscles; and infections. In addition to potential pathologies affecting muscle tissues, aging progressively affects muscular system as lean body mass naturally decreases over time, and this decrease is partly due to the loss of muscle tissue (atrophy).

The rate and severity of muscle changes may be caused by genetics, often beginning at age 20 in men and 40 in women. Muscles are less toned and less able to contract due to normal changes in muscle tissue and changes in the nervous system with aging, leading to stiffness, reduced endurance and strength. Muscle weakness also contributes to fatigue and decreased tolerance for physical activity. Joint problems ranging from mild stiffness to debilitating arthritis (osteoarthritis) are very common.

In mammals, aging is associated with a reduction in skeletal muscle mass and function termed sarcopenia. Sarcopenia in humans means a reduced ability to perform activities of daily living, resulting in a loss of independence. In fact, low skeletal muscle mass or strength is reported to be the most common cause of disability in the elderly (Fielding, 2011, Potes et al., 2019) and is a predictor of morbidity, loss of independence, frailty, and mortality, independent of other risk or disease factors (Nair, 2005).

Sarcopenia is characterized by atrophy of muscle fibers, showing a greater decrease in type 2 fast twitch fibers compared to type 1 slow twitch fibers, with an increase in fiber size heterogeneity and non-contractile tissues (adipose and connective) within muscles (Romanick et al., 2013). Effective treatment of skeletal muscle atrophy is difficult due to inadequate understanding of its underlying biological mechanisms. As in obesity, one of the factors that may contribute to the pathogenesis of sarcopenia is oxidative stress (Scicchitano et al., 2018). It is still controversial whether insulin resistance appears as a direct consequence of age (Oya, 2014; Soriguer, 2014); however, evidence show that reduction in lean body mass and the relative increase in fat mass (also known as sarcopenic obesity), directly contributes to insulin resistance in the elderly (Srikanthan et al., 2010). Recent reports indicate that muscle mitochondrial function declines with aging (Theurey and Pizzo, 2018, Del Campo et al., 2018), and this age-associated decline in mitochondrial function contributes to insulin resistance in elderly men (Chow, 2018).

The inventors of the present application have made an important contribution in recent years to the understanding of the cellular mechanisms involved in the adaptation of skeletal muscle to exercise; in particular, they have described a mechanism that relates electrical stimulation to changes in gene expression (Casas et al., 2014), depending on the sequential activation of Cav1.1 (or DHPR) as a voltage sensor and Pannexin-1 channel (Panx1) as a pathway for ATP egress from the muscle cell that is followed by activation of purinergic P2Y receptors, leading to different signaling cascades that influence gene expression (such as IL-6, Bustamante et al., 2014), metabolism and redox homeostasis (Araya et al., 2003, Buvinic et al., 2009, Casas et al., 2010, Jorquera et al., 2013, Diaz-Vegas et al., 2018).

Alongside, the signaling pathway described for normal young adult skeletal muscle appears to be substantially altered in conditions such as muscular dystrophy, aging, and obesity (Valladares et al., 2013, Altamirano et al., 2013, Del Campo et al., 2018) and it is likely that these variations are involved in the phenomenon of muscle wasting characteristic of dystrophy, sarcopenia and sarcopenic obesity (Kalinkovich and Livshits, 2017). In addition, ATP is involved in the activation of NADPH oxidase 2 (NOX2) in skeletal muscle (Diaz-Vegas et al., 2015) and reactive oxygen species (ROS) are important second messengers in skeletal muscle at low concentrations, involved in gene expression, insulin-dependent glucose transport and mitochondrial biogenesis, among other processes (Henriquez-Olguin et al., 2015, 2019). At higher concentrations, on the other hand, ROS are mediators of obesity-related phenotypes, including insulin resistance and diabetic skeletal muscular atrophy, as well as muscle atrophy in aging (Espinosa et al., 2016).

In the signaling pathway described in normal young muscle, the skeletal muscle fiber releases ATP upon electrical stimulation, which constitutes a physiological signal for gene expression leading to muscle adaptation (Jorquera et al., 2013). ATP release occurs through Panx1 channels (Jorquera et al., 2013) and can act on P2Y purinergic receptors to trigger intracellular signaling cascades in skeletal muscle (Buvinic et al., 2009, Diaz-Vegas et al., 2015), including activation of PI3 kinase, phospholipase C, and production of diacylglycerol (DAG) and IP3 (Eltit et al., 2006). The Panx1 channel is regulated in the plasma membrane by its functional interaction with the voltage sensor, Cav1.1 (Arias-Calder6n et al, 2016, Jaque-Fernindez et al., 2021). Indeed, pharmacological inhibition of Cav1.1 with nifedipine inhibits ATP release elicited by electrical stimulation in normal muscle fibers (Jorquera et al., 2013).

Extracellular ATP, via activation of PKC by DAG and calcium, induces the production of ROS through the activation of NADPH oxidase (NOX-2 (Diaz-Vegas et al., 2015)) and NOX-2 is the main source of ROS during exercise in skeletal muscle (Henriquez-Olguin et al., 2019). It is important to note that both extracellular ATP and ROS are involved in inflammatory responses (Kawamura et al., 2012). NOX-2-dependent ROS production is involved in NFκB activation and nuclear translocation in skeletal muscle fibers (Diaz-Vegas et al., 2015).

Importantly, the healthy physiologic process described in skeletal muscle is greatly disrupted in some pathologic conditions. Indeed, basal ATP release through Panx1 channels is greatly increased in muscle fibers from dystrophic mice (Valladares 2013), aged mice, and mice fed a high-fat diet (Jorquera et al, 2021).

Dystrophic mice are characterized by a progressive and severe loss of muscle fibers due to cell death, inflammation, and oxidative stress (Altamirano, et al., 2013). In its muscle fibers, excess extracellular ATP is pro-apoptotic, inducing Bax, BIM, and PUMA transcription and increasing activated Bax levels (Valladares, 2013). Importantly, nifedipine treatment can normalize excess basal ATP release in dystrophic muscle fibers, in vitro (Valladares et al., 2013) and in vivo (Altamirano et al., 2013), where a daily injection of this drug for a week reduced the mRNA levels of pro-oxidative (p47(phox) NOX2 subunit) and pro-apoptotic (Bax) genes, reduced creatinine levels serum kinase (a marker of muscle damage) and improved muscle function (Altamirano, 2013). This point is very relevant due to the similarities with the models of sarcopenia and obesity. In fact, it has been found that basal extracellular ATP levels are also increased in obese and aged mice, and that nifedipine treatment also improves muscle function in these two models. It seems, then, that there is a common pathway underlying muscle wasting and dysfunction and that it depends on an excessive release of ATP from muscle fibers for at least these three muscle conditions: dystrophy, sarcopenia, and obesity.

To date, no effective treatment has targeted skeletal muscle to stop wasting away and restore function. Understanding the pathways that lead to muscle dysfunctions offers the possibility of finding new therapeutic targets for these conditions. The expression of proinflammatory genes can be substantially decreased using a Panx1 channel blocker (Jorquera et al., 2021).

Since Cav1.1 activation is upstream of ATP release through Panx1 channels in skeletal muscle fibers, and Cav1.1 blockers such as nifedipine can modulate ATP release, the inventors of present application have designed a drug (MP-2) that interacts with Cav1.1 channels in skeletal muscle, but not in arterial smooth muscle, to inhibit ATP release from muscle fibers without producing hypotensive side effects in patients.

### STATE OF THE ART

Muscular dystrophy is a group of diseases that cause progressive weakness and loss of muscle mass. In muscular dystrophy, abnormal genes (mutations) interfere with the production of proteins needed to build healthy muscles. There are many types of muscular dystrophy, with symptoms that might begin at early ages, mainly in males, while other types do not appear until middle age or later. All forms of muscular dystrophy worsen as muscles weaken, and most people with this condition eventually lose the ability to walk.

There is no cure for muscular dystrophy, however medication and treatment can help control symptoms and slow the progression of the disease. This includes physical and speech therapy, orthopedic devices, surgery, and medications. Some people with muscular dystrophy have mild cases that slowly get worse; other cases are disabling and severe.

Sarcopenia is the loss of muscle mass that occurs with aging. Thus, muscle mass is gradually lost from the age of 30, accelerating the process from the age of 60, which may lead to disability and lack of independence in patients, alongside a major risk of falls and fractures or injuries. Ultimately, as body muscle mass reduces, fatty tissue increases, resulting in serious risk of developing hypertension, obesity, or diabetes. Symptoms of sarcopenia are low muscle mass or gradual loss, decreased muscle strength, and reduced physical performance.

People who suffer from this disease usually present a feeling of weakness, with difficulty getting up from a chair or slow walking speed being common, along with repeated falls. It is also common for them to describe recent weight loss without just cause or loss of muscle mass. In the long run, development of this pathology can be associated with adverse consequences such as fractures, physical disability, increased hospital admissions, worsening of quality of life, and mortality.

The main cause of sarcopenia is the passage of time and aging, since several factors interact in this process: genetic, hormonal, lifestyle changes, weight loss, and loss of spinal cord motor units. Physical activity and an active lifestyle can prevent and delay the onset and progression of sarcopenia, leading to greater longevity and independence of patients.

Therapeutic approaches to sarcopenia can be done through pharmacological and non-pharmacological measures. Since there is no specific treatment for this pathology, some methods are focused on delaying its onset:
Testosterone treatment: used in patients with low levels of testosterone in the blood since testosterone is related to the presence of muscle mass and strength in the legs.
Growth hormone treatment: This method can increase muscle mass and strength in people with hypopituitarism.
Nutritional treatment: high protein diets and other specific nutrients provide strength and decrease the risk of sarcopenia progressing.
Physical exercise: training and physical exercise is key to counteracting sarcopenia, improving strength, endurance, and balance. It is necessary for the patient to carry out a training routine of several weeks, which is very effective in increasing muscle mass and the patient's independence.
Interventions in the immune system: in some cases, substances such as pentoxifylline are used to regulate the production of cytokines, a substance that causes the loss of muscle mass.
Use of cytokines (myokines) or myokine receptor agonists is in the experimental stage.

The state of the art describes a series of compositions, formulations and procedures for obtaining compounds of the dihydropyridine type and some of tetrahydrofuran[3,4-b]pyridines, including homologous compounds. Said documents establish the homologous properties and mechanisms of action based on their action on dihydropyridine receptors. Methods of synthesizing homologous compounds are further described. However, said documents identify the use of these compounds for the treatment of other diseases such as cardiovascular diseases.

Within the state of the art, the following patent documents are particularly relevant:

The document U.S. Pat. No. 4,532,248 (A) refers to compounds derived from 1,4-Dihydropyridines of the formula

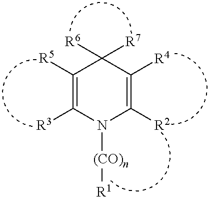

wherein:
n is 0, 1 or 2, and
and R¹ to R⁷ may have a wide variety of meanings, possessing inotropic action, and many of which are new and useful in increasing Ca entry in cells, particularly to combat coronary and vascular diseases, hypertension, swelling in the mucous membranes, and diseases involving increased blood sugar or an incorrect balance of salt and fluids.

This document discloses (at Example 5) a compound having the following chemical structure is:

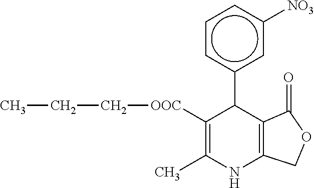

2-methyl-4-(3-nitrophenyl)-5-oxo-1,4-dihydropyridine-5,7-dihydrofuo [3,4-b]pyridine 3-carboxilato.

However, such compounds disclosed in U.S. Pat. No. 4,532,248 (A) have long-acting vasodilating and antihypertensive action, thus not recommended to be used in the treatment of muscular disorders.

Document WO 2009144450 Al discloses a compound of formula (1):

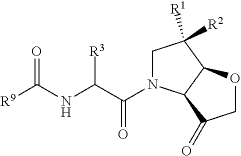

or a pharmaceutically acceptable salt, hydrate, complex or prodrug thereof, wherein one of R¹ and R² is H, and the other is selected from alkyl C₁₋₈, cycloalkyl C₃₋₆ and C₁₋₈-alkyl-C₅₋₁₀-aryl; R³ is selected from tert-butyl, cyclopentyl and 1-methylcyclopentyl; R⁹ it is selected from different compounds detailed in the patent application.

The invention further relates to pharmaceutical compositions comprising compounds of formula (I), and to the use of said compounds in the treatment of various diseases such as osteoporosis, Paget's disease, Chagas disease, malaria, gingival diseases, hypercalcemia, metabolic bone disease, diseases involving matrix or cartilage degradation and bone cancer disorders such as bone metastases and associated pain.

However, this document differs from the invention of the present application in that it does not disclose compounds possessing the structure dihydropyridines, nor compounds derived from the tetrahydrofuran[3,4-b]pyridine nucleus.

Structurally, the present invention with the document WO 2009144450 Al differs substantially in the pharmacophore group that makes up each invention, meaning that the molecules of both the present invention and the cited document aim for different therapeutic targets.

Accordingly, there is a need for new pharmaceutical compositions useful in the prevention and treatment of dystrophy and sarcopenia. This patent application aims to address the technical problem of providing an efficient and safe drug capable of improving muscle function in elderly patients, with low or no side effects on vasoconstriction.

More specifically, the present application aims to provide a treatment for muscle weakness in older adults, which to date has not been solved, by providing derivatives of the tetrahydrofuran[3,4-b]pyridine nucleus with receptor binding capacity of dihydropyridine due to its chemical structure that does not affect the current of Ca⁺² channels which allows to safely treat sarcopenia, decreasing the cardiovascular effects that can trigger the Ca⁺² channel.

### SUMMARY OF THE INVENTION

The present invention relates to compounds derived from tetrahydrofuran[3,4-b]pyridine-3-carboxylate, pharmaceutical compositions comprising said compounds, therapeutic use of said compositions in prevention and treatment of muscle disorders in a subject in need thereof, and a method of prevention and treatment of muscle disorders comprising administration of said compositions for recovery of muscle function in patients suffering from dystrophy and sarcopenia.

It is an object of the present invention to provide compounds and pharmaceutical compositions derived from DHP, with few or no side effects on vasoconstriction and high therapeutic potential for muscle weakness and muscular disorders, like dystrophy, cachexia and/or sarcopenia, that are able to increase muscle strength and endurance in a subject in need thereof.

### DESCRIPTION OF THE FIGURES

FIG. 1 shows a 1H proton nuclear magnetic resonance spectrum for MP2.

FIG. 2 shows a ¹³C carbon nuclear magnetic resonance spectrum for MP2.

FIG. 3 shows the infrared spectrum of MP2.

FIG. 4 shows the effect on blood vessel contraction of a selected dihydropyridine (nifedipine) compared to MP-2.

FIG. 5 shows basal ATP release from isolated muscle fibers as a function of age.

FIG. 6 shows the inhibition of ATP release by electrical stimulation in muscle fibers.

FIG. 7 shows muscle strength in aged mice as a function of time.

FIG. 8 shows how MP-2 treatment improves both strength and muscular endurance.

FIG. 9 shows that extracellular ATP contents can induce the expression of proinflammatory genes.

### DETAILED DESCRIPTION OF THE INVENTION

According to the research carried out by the inventors, it has been shown that the use of molecules from the dihydropyridine (DHP) family that target the dihydropyridine receptor (or Cav1.1 calcium channel) normalize gene expression in muscle fiber and functional parameters of dystrophic and sarcopenic muscles.

In accordance with the foregoing, this patent application discloses compounds derived from tetrahydrofuran [3,4-b]pyridine-3-carboxylate able to increase muscle strength and endurance in subjects and patients suffering from dystrophy and sarcopenia.

The disclosed compounds have the following chemical structure:

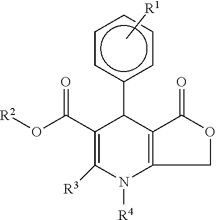

wherein:
R¹ is a nitro group;
R² is hydrogen or alkyl C₁-C₃;
R³ is hydrogen or alkyl C₁-C₃;
R⁴ is hydrogen, or
a pharmaceutically acceptable salt thereof.

In one embodiment of the invention, the compound is selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridine-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridin-3-carboxylate;
propyl 1,2-propyl-4-(6-nitrophenyl)-5-oxo-1,4,5,7-tetrahydrofuro [3,4-b]pyridine-3-carboxylate.

The present application also discloses a pharmaceutical composition comprising the compounds derived from tetrahydrofuran [3,4-b]pyridine-3-carboxylate having the chemical formula:

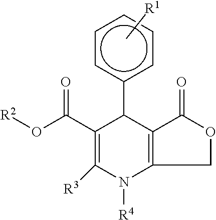

wherein:
R¹ is a nitro group;
R² is hydrogen or alkyl C₁-C₃;
R³ is hydrogen or alkyl C₁-C₃;
R⁴ is hydrogen, or
a pharmaceutically acceptable salt thereof;
and a pharmaceutically acceptable carrier.

In one embodiment, said pharmaceutical composition comprises a compound selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridine-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridin-3-carboxylate;
propyl 1,2-propyl-4-(6-nitrophenyl)-5-oxo-1,4,5,7-tetrahydrofuro [3,4-b]pyridine-3-carboxylate.

The present application also discloses a pharmaceutical composition for prevention or treatment of a muscle disorder in a subject in need thereof comprising a compound having the chemical formula:

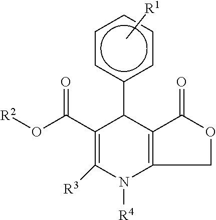

wherein:
R¹ is a nitro group;
R² is hydrogen or alkyl C₁-C₃;
R³ is hydrogen or alkyl C₁-C₃;
R⁴ is hydrogen, or
a pharmaceutically acceptable salt thereof;
and a pharmaceutically acceptable carrier.

In one embodiment, said pharmaceutical composition comprises a compound selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridine-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridin-3-carboxylate;
propyl 1,2-propyl-4-(6-nitrophenyl)-5-oxo-1,4,5,7-tetrahydrofuro [3,4-b]pyridine-3-carboxylate.

In another embodiment, said muscle disorder is selected from the group consisting of cachexia, sarcopenia or muscular dystrophy.

In another embodiment, said compositions are administered via intravenous, intramuscular, transmucosal, intrathecal, systemic, intraperitoneal, ophthalmologic and/or subcutaneous manner to a subject in need thereof.

In another embodiment, said subject is a mammalian. In a further embodiment, mammalian is selected from human, dog, cat and horse.

Finally, the present application also discloses a method for prevention or treatment of a muscle disorder, comprising administering a therapeutically effective amount of a pharmaceutical composition comprising a compound having the chemical formula:

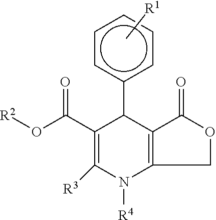

wherein:
R¹ is a nitro group;
R² is hydrogen or alkyl C₁-C₃;
R³ is hydrogen or alkyl C₁-C₃;
R⁴ is hydrogen, or
a pharmaceutically acceptable salt thereof;
and a pharmaceutically acceptable carrier.

In one embodiment, said pharmaceutical composition comprises a compound selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridine-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridin-3-carboxylate;
propyl 1,2-propyl-4-(6-nitrophenyl)-5-oxo-1,4,5,7-tetrahydrofuro [3,4-b]pyridine-3-carboxylate.

In another embodiment, said muscle disorder is selected from the group consisting of cachexia, sarcopenia or muscular dystrophy.

In another embodiment, said compositions are administered via intravenous, intramuscular, transmucosal, intrathecal, systemic, intraperitoneal, ophthalmologic and/or subcutaneous manner to a subject in need thereof.

In another embodiment, said subject is a mammalian. In a further embodiment, mammalian is selected from human, dog, cat and horse.

### EXAMPLES

The inventors have established the crucial role of the dihydropyridine receptor (DHPR, L-type calcium channel, Cav1.1) as a sensor of depolarization of the adult muscle fiber membrane, allowing the activation of signals that regulate gene expression. The release of ATP via Pannexin-1 (Panx1) channels after electrical stimulation of the muscle fiber plays a key role as a mediator of this process.

Activation of the Panx1 channel produces the release of ATP from the muscle fiber with the consequent activation of P2Y-type purinergic receptors present in the fiber membrane. This signaling cascade is the beginning of a series of processes that end with changes in gene expression. The crucial role of DHPR in triggering this process has been demonstrated using 1,4 dihydropyridines (DHP), molecules capable of binding to DHPR and modulating its function. Nifedipine, a DHP antagonist of DHPR that produces complete inhibition of Ca²⁺ current through the channel, produces almost total inhibition of ATP outflow after electrical stimulation of the muscle fiber. On the other hand, the use of (S)-(−)-BayK-8644, a DHP agonist of DHPR that increases Ca²⁺ current through the channel, also inhibits ATP release, as does Nifedipine. Together these results allowed the inventors to postulate a new function for DHPR, different from its function as a Ca²⁺ channel and Ryanodine channel activator during muscle contraction (EC coupling).

This function is linked to the activation of the Panx1 channel after electrical stimulation of the muscle fiber at certain frequencies, which induces the release of ATP into the extracellular medium and the consequent activation of signaling pathways that produce changes in gene expression. In addition, and consistent with the results presented above, myotubes lacking the DHPR (product of KO mice for the gene that forms the Ca²⁺ channel pore) did not show ATP release after being electrically stimulated, but do present abnormally high extracellular ATP basal levels, indicating that DHPR has a central role in both processes: activating ATP release after electrical stimulation and maintaining low levels of extracellular ATP under resting or basal conditions. The importance of the relationship between DHPR and ATP release by the Panx1 channel is reinforced by results showing that DHPR and Panx1 co-localize in the T tubules of muscle fibers.

The described excitation-signaling coupling mechanism involving the DHPR-Panx1-ATP release axis is found to be altered in various muscle pathologies. In particular, the inventors have studied the status of this mechanism in sarcopenic mouse models. In this model, findings appear such as: high basal levels of extracellular ATP, aberrant response to electrical stimulation of these fibers, altered expression of pro-inflammatory and pro-apoptotic genes (preliminary results).

The results show that treatment with the new drug MP-2 in aged mice (age>18 months) substantially improves their muscle strength.

It is important to note that the 1,4 DHP family molecules influence the cardiovascular system due to their action on Ca²⁺ currents through the channel formed by DHPR in the muscle membrane. In this sense, MP-2 is an advance towards a safe treatment of sarcopenia, since it is a molecule designed based on 1,4 DHP, in order to ensure its binding to DHPR, but taking care to reduce its inhibitory or activating effect of the Ca²⁺ current through this channel, to reduce its cardiovascular effects.

The result is a molecule with a chemical base like (but not one of) DHPs, which has a minimal effect on Ca²⁺ currents through the channel and, above all, minimal effects on the inhibition of vasoconstriction.

### Example 1

### Synthesis of the compound methyl 2-methyl-4-(2-nitrophenyl)-5-oxo-1,4,5,7-tetrahydrofuro [3,4-b]pyridine-3-carboxylate (MP2)

The MP2 compound was synthesized according to the synthetic routes described in the publication “Methods for the synthesis of 4-pyrazolyl- and 4 pyridyl-5-oxo-1,4,5,7-tetrahydrofuro[3,4-b]pyridines”, Chemistry of Heterocyclic Compounds volume 31, pages 841-846 (1995) and, in U.S. Pat. No. 4,567,268 A (Process for preparation of certain tetrahydrofuro[3,4-b]pyridines).

### Physicochemical characterization of methyl 2-methyl-4-(2-nitrophenyl)-5-oxo-1,4,5,7-tetrahydrofuro [3,4-b]pyridin-3-carboxylate (Molecular Formula “C16 H14 N2 O6”)

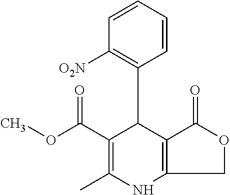 [SMILES: COC(=O)C1=C(C)NC2=C(C(=O)OC2)C1c1ccccc1[N+](=O)[O-]]

Molecular weight
330.29 grams/mol
Melting point (experimental)
Value: 259-262° C. I Condition: Solvent: acetone (67-64-1); hexane (110-54-3)
Boiling point (predicted)
Value: 545.5±50.0° C.| Condition: Press: 1 atm (760 Torr)
Density (predicted)
Value: 1.44±0.1 g/cm³| Condition: Temperature: 20° C. Press: 1 atm (760 Torr)
pKa (predicted)
Value: −1.47±0.701 Condition: Most basic temperature: 25° C.

### Spectrum Analysis/Signal Identification:

I) The [¹H]proton spectrum analysis was performed using the Bruker TopSpin 3.5 pl2 program. For the sample 11 signals are observed.

¹H NMR (300 MHz, DMSO, TMS, 25° C.) δ: 9.95 (s, 1H, Ha), 7.81 (dd, 1H, Ar-Hb), 7.65 (td, 1H, Ar-Hc), 7.46 (dd, 1H, Ar-Hd), 7.41 (td, 1H, Ar-He), 5.52 (s, 1H, Hf), 4.92, 4.83 (quartet AB, 2H, Hg, Ja=16.18 Hz, Jb=16.11 Hz), 3.37 (s, 3H, Hh),3.32 (s, H2O), 2.5 (s, DMSO), 2.3 (s, 3H, Hi).

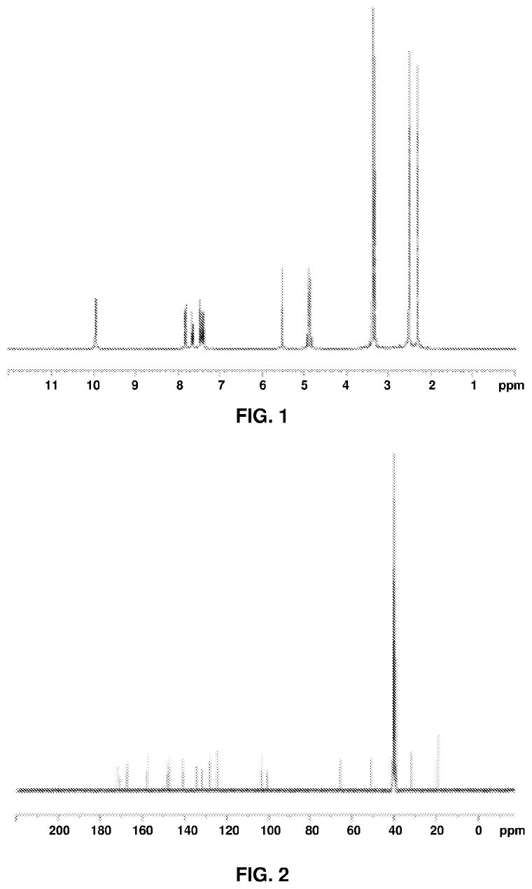

The schematic of molecule 1 is shown below and the 1H proton nuclear magnetic resonance spectrum is shown below and in FIG. 1.

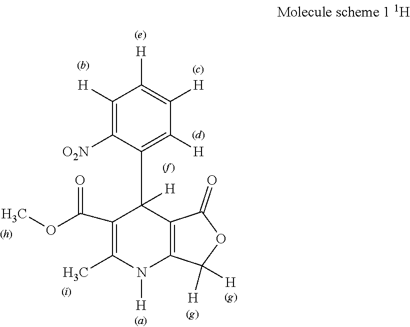 [SMILES: COC(=O)C1=C(C)NC2=C(C(=O)OC2)C1c1ccccc1[N+](=O)[O-]]

II) Carbon 13 spectrum analysis [13C] was performed using the Bruker TopSpin 3.5 pl2 program. For the sample 1 7 signals are observed.
¹³C NMR (75.47 MHz, DMSO, TMS, 25° C.) δ: 171.3 (C1), 167.1 (02), 157.5 (C3), 148.3 (C4), 147.3 (C5), 140.8 (C6), 133.9 (C7), 131,8 (C8), 128.0 (C9), 124.0 (C10), 102.9 (C11), 100.6 (C12), 65.7 (C13), 51.3 (C14),39.98 (DMSO), 32.2 (C15), 19.5 (C1 6).

The schematic of molecule 2 is shown below and the 13C nuclear magnetic resonance spectrum is shown in FIG. 2.

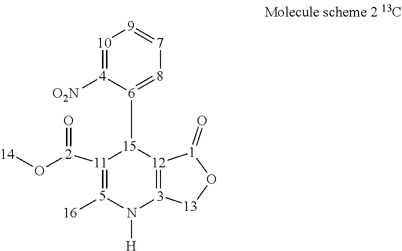

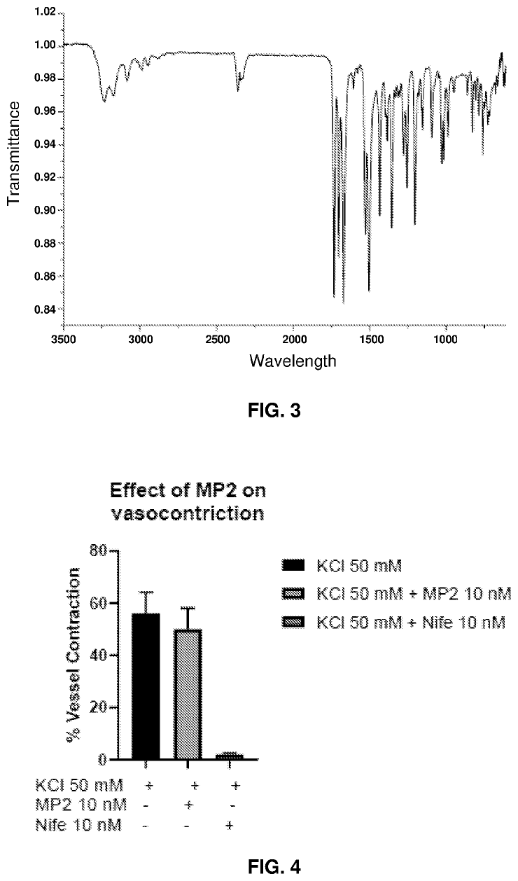

III) Infrared spectroscopy analysis is shown in FIG. 3: ATR-FTIR (cm-i) 3232(v NH), 1731 (v C═O lactone), 1701 (v C═O ester), 1525 (v asymmetric NO), 1354 (v symmetric NO). *2359 (v C═O residual 002).

According to the analyzes performed, in one and two dimensions through nuclear magnetic resonance spectroscopy, the complete assignment of protons and carbon of the methyl 2-methyl-4-(2-nitrophenyl)-5-oxo-1,4,5,7 tetrahydrofuro [3,4-b]pyridine-3-carboxylate molecule was performed.

### Example 2

### Isolation of Adult Skeletal Muscle Fibers

Isolation of skeletal muscle fibers was performed from C57BL/J6 mice between 8 and 10 weeks of age. Isolated fibers were obtained from the flexor digitorum brevis (FoB) and Gastrocnemius muscle by enzymatic digestion for 90 min with 400 U/mL type II collagenase (Worthington Biochemicals Corp., Lakewood, NJ, USA) and mechanical dissociation with Pasteur pipettes, as previously described (Casas et.al, 2010). The isolated fibers were seeded in Petri dishes coated with a layer of Matrigel in Dulbecco's modified Eagle's culture medium (DMEM), supplemented with 10% horse serum, 50 U/mL penicillin and 50 mg/mL streptomycin. Cell cultures were used approximately 20 h after the fiber seeding process. All protocols were previously approved by the Bioethics Committee of the Faculty of Medicine of the University of Chile.

### Example 3

### Electrical Stimulation of Muscle Fibers In Vitro

Isolated skeletal muscle fibers were stimulated with platinum electrodes connected to an electro-stimulator (Grass S48; W. Warwich, RI, USA). The protocol consisted of the application of 270 square pulses of 0.3 ms duration each at a frequency of 20 Hz, as previously described (Jorquera et al. 2013). During the stimulation experiments, the fibers were maintained in Dulbecco's modified Eagle's medium (DMEM) supplemented with 10% horse serum, 50 U/mL penicillin and 50 mg/mL streptomycin. In addition, the fibers were incubated with 50 pM N-benzyl-P-toluenesulfonamide (BTS, Sigma-Aldrich) to inhibit muscle contraction. All experiments were performed at room temperature of 21-23° C.

### Full Cell Voltage-Clamp: Measurement of L-Type Ca²⁺ Currents

An electrophysiology kit consisting of a confocal microscope (Zeiss, Pascal 5), a Digidata 1322A amplifier (Axon Instrument, Austin, TX) in whole cell configuration was used for this assay. To generate the command pulse and for data acquisition, the Clampex 9.2 software led to an A/D, D/A converter (Axon Instruments, Austin, TX) was used.

Measurements of Ca²⁺ currents were performed on single skeletal fibers isolated from C57 mouse FDB muscle (Collet, 2004; Pouvreau, 2007). One fiber was used as a control against itself due to the variability observed between muscle fibers. Borosilicate capillaries were used and microelectrodes with a resistance between 2-5 MO were generated, which is an adequate size for diffusion and to enter the cell without destroying it, as described in the literature (Pouvreau, 2007; Hernández-Ochoa, 2012).

Single fibers were isolated with silicone, in order to perform a whole cell controlled potential assay on a small portion of the fiber end.

Prior to this, the bottom of the plate was coated with a thin layer of industrial silicone (previously proven not to cause damage or cell death) in a homogeneous manner, after which the muscle fibers obtained by mechanical disintegration were deposited directly in DMEM solution with 10% horse serum. Fibers were loaded with 20 mM EGTA for 30 minutes. Next, between 5-10 fibers per plate were siliconized with a micro-syringe containing industrial silicone in a less liquid state that the one used in the base of the plate in 80% of the extension of said fibers, leaving a small portion free to be used under a microscope.

Once the fibers have been siliconed on a plate, the DMEM solution (10% FBS) were replaced by the external solution used for the Ca²⁺ Current measurement experiment which is described below. Whole cell controlled potential assay was performed with a microelectrode filled with internal solution containing (in mM): 140 Potassium glutamate; 5 Na2-ATP; 5 sodium phosphocreatine; 5.5 MgCl₂; 5 D-glucose, 5 HEPES, adjusting to pH 7.2 with KOH. The external solution contains (in mM) 140 tetraethylammonium methanesulfonate (TEA); 2.5 CaCl₂); 2MgCl₂; 0.002 tetrodotoxin (TTX); 1 4-aminopyridine (4-AP); 10 HEPES, adjusting to pH 7.2. EGTA is used in the internal solution as a Ca²⁺ chelator to prevent intracellular levels of Ca²⁺ free rise to points that generate muscle contraction. TEA and 4-AP are used in the external solution to block voltage-gated and non-gated K⁺ channels. TTX is a toxin that selectively blocks voltage-gated Na⁺ channels (Pouvreau, 2007; Hernández-Ochoa, 2012).

For each experiment, a different microelectrode was used whose resistance was checked each time when entering the external solution. Next, the microelectrode entered through the silicone, in the isolated part of the fiber. Analog compensation was made in the amplifier (the current for the load is increased and thus the time constant is decreased) to reduce the effect of the series resistance. Past 20 minutes after entering the cell and diffusion of EGTA, the current of Ca²⁺ through Cav1.1 was recorded with the cell controlled at a basal potential of −80 mV. Starting from this basal potential, command pulses of 500 millisecond duration were applied from −40 mV to 80 mV, with a scaled increase in amplitude of 10 mV, applied every 30 seconds. This protocol was performed 3 times per fiber, with intervals of 5 min. Each command pulse was preceded by a −20 mV hyperpolarizing prepulse which is the same for all voltages.

Linear leakage component of the current was eliminated by subtracting a scaled value from the measured current generated by the prepulse, using the P/1 protocol.

All experiments were performed at a room temperature of 20-22° C.

Effect on L-Type Currents To ensure that MP-2 interacts with Cav1.1 channels (dihydropyridine receptors), L-type calcium currents were measured in isolated fdb muscle fibers treated with a 10 μM concentration of five different dihydropyridines (nifedipine, nitrendipine, nicardipine, nimodipine and nisoldopine) or with 10 μM MP-2 (Table 1). DHPs had an effect ranging from 14.3% to 83% inhibition of the current and MP-2 produced 50±8% inhibition, therefore demonstrating that Cav1.1 channels are a target of MP-2.

TABLE 1
Effect of dihydropyridines and MP-2 on L-type calcium
peak current in mouse skeletal muscle fibers

| Drug used | | | | | | |
|---|---|---|---|---|---|---|
| (10 μM) | Nifedipine | Nicardipine | Nitrendipine | Nimodipine | Nisoldipine | MP-2 |
| % inhibition of | 83 ± 13 | 32.6 ± 8 | 47.6 ± 2 | 48 ± 10 | 14.3 ± 7 | 50 ± 8 |
| Ca²⁺ current | | | | | | |

At least 3 fibers were used for each drug tested (from 2-3 different mice). The control in each condition is the maximum current in the absence of a drug.

### Example 4

### Vasoconstriction

### MP-2 has Low Effects as a Vasodilator

Diameter of the pulmonary vessels was measured in thin slices of rat lung using microscopy as described by Henriquez et al. 2018.

Some DHP derivatives may induce a strong vasodilator effect, representing an undesirable side effect in treatment of muscular disorders. FIG. 4 shows results of an ex vivo vessel contraction test where MP-2 inhibited only 10% of the contraction in pulmonary vessels induced by depolarization with 50 mM KCl, while a standard DHP (Nifedipine) inhibits contraction under the same conditions between 90-100%.

As a low vasoconstrictor effect of MP-2 needs to be controlled, the effect of various dihydropyridines in addition to MP-2 on high potassium-induced contraction of pulmonary vein rings was tested. FIG. 4 shows the effect on blood vessel contraction of a selected dihydropyridine (nifedipine) compared to that of MP-2. FIG. 4 shows the mean inhibitory effect±SEM of the percentage of vasoconstriction induced by nifedipine (close to 100% inhibition of contraction), whereas the contractile response in the presence of MP-2 was less than 10% inhibition (n=7 blood vessels) compared to control vessels without the drug.

### Example 5

### Inhibition of ATP Release

### Determination of Extracellular ATP Concentration by Luciferin/Luciferase Assay.

Extracellular ATP concentrations were determined using the CellTiter-Glo® Luminescent Cell Viability Assay Kit (Promega). 25 μL of extracellular medium was obtained from DAGe and DAGne cultures at different times (from 0 to 900 seconds). 25 μL of reconstituted reagent (buffer substrate) was added. After incubating for 10 min in the dark at room temperature, the samples were quantified in an FB12 (Berthold) luminometer. Alongside, a standard curve was made from 1 fmol to 100 pmoles of ATP (Sigma Aldrich) using the same kit protocol. The values obtained for the samples were interpolated in the standard curve to determine the ATP concentrations in each condition. The results obtained were normalized in pmol of extracellular ATP per pg of total RNA in each plate. RNA normalization was preferred over total protein normalization since adult fiber cultures were seeded on a matrigel sheet, which has a large amount of protein and could alter the quantification process.

Basal ATP Release is Greatly Increased in Muscle Fibers from Aged Mice.

Basal extracellular ATP levels (condition without electrical stimulation) were measured in fdb muscle fibers from mice of different ages. Basal ATP levels are strongly elevated in fibers from mice aged 16 months and older. N=5 for each age. The mean±SEM is plotted.

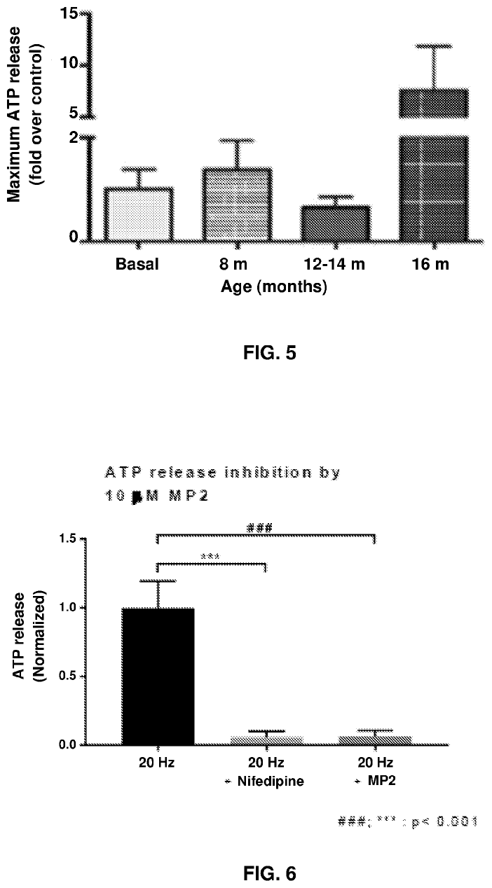

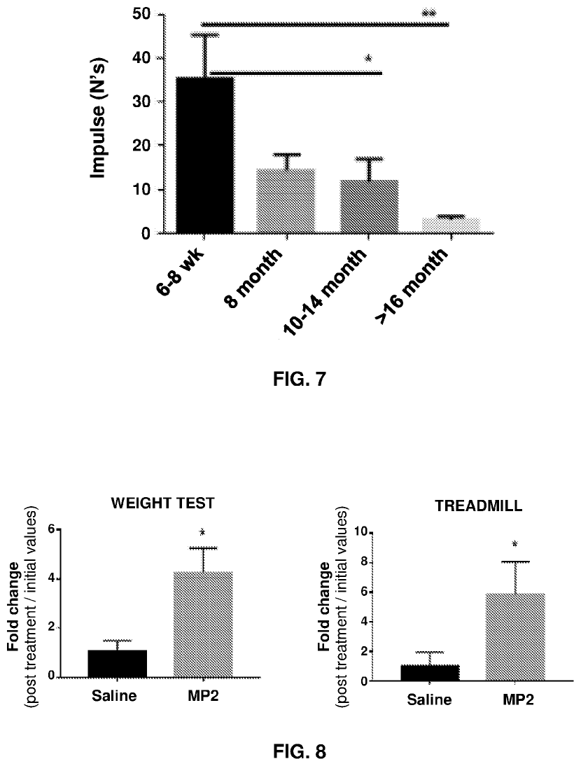

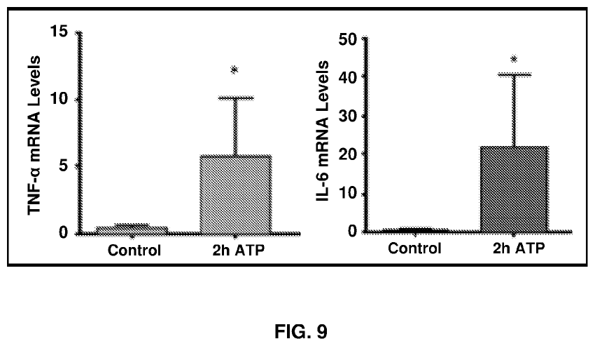

FIG. 5 shows the basal ATP release from isolated muscle fibers as a function of age. The old mice (16 months old) have a much higher release compared to the younger mice. ATP release is greatly increased in muscle fibers of aged mice.

### Example 6

### MP-2 Inhibits ATP Outflow Induced by 20 Hz Electrical Stimulation in Muscle Fibers

Pre-incubation of fdb muscle fibers with MP-2 at 10 μM for 30 min inhibits the outflow of ATP that is triggered after electrically stimulating muscle fibers at 20 Hz. Maximum ATP release is observed in electrically stimulated control fibers (black bar), inhibition obtained by pre-incubation of the fibers with nifedipine (light gray bar) and inhibition achieved with MP2 (dark gray bar). It is observed that MP2 inhibits in a similar way as nifedipine. ####: p<0.001 (20 Hz) vs (20 Hz MP-2). (N=5). The mean±SEM is plotted. ****: p<0.001. Data are shown as fold change over control (no drug, stimulated at 20 Hz).

Isolated mouse fibers of the fdb muscle were electrically stimulated at 20 Hz in the presence of either nifedipine or MP-2. As previously published, nifedipine almost completely blocks ATP release and the effect of MP-2 here is indistinguishable from that of nifedipine.

### Example 7

### Functional Effects

### Muscle Strength Decreases Sharply in Aged Animals

Muscle strength in mice at different ages was measured with an inverted grip test and reported as Impulse (N*s), which considers the amount of time each mouse supports its own body weight in this test. A decrease in Impulse is observed from 8 months of age and it decreases drastically in mice older than 16 months. N=4 for each age. Mean±SEM * is plotted: p<0.05; **: p<0.01. Muscle strength is greatly diminished in aged mice.

### Example 8

Aged Mice Treated with 1 mg/Kg MP-2 Per Day for 12 Days Improve Muscle Function

The standardization of this test that evaluates resistance and strength was carried out in the treadmill equipment for rodents. The test consists of 5 sessions where the animals were acclimatized for 5 minutes at the equipment's minimum speed of 0.3 Km/h and the number of shots of compressed air that keep the animal trotting is counted. Once the acclimatization process was finished, the animals were evaluated in an incremental test consisting of 2-minute ramps at the minimum speed and then gradually increasing 0.1 Km/h after 2 minutes until the animal could no longer run. This maximum effort test is only performed once per session and is considered finished when the animal reaches the bottom of the ramp and accumulates 10 shots of compressed air in a period of 10 seconds.

The muscle function of aged mice (between 19 and 21 months of age) was assessed with an incremental strength test (weightlifting) and with an incremental treadmill running test. The measurement was made after 12 days of treatment with MP-2 (1 mg/Kg/day) administered via intraperitoneal injection. As a control, vehicle-injected mice were used in the same manner as drug-injected mice. Recovery of muscle function can be observed after treatment. Data were analyzed as the change in test performance at the end of treatment relative to performance at the start of treatment for each mouse. It is observed that the treatment improves the strength parameters more than 4 times with respect to the control. The Endurance test (incremental treadmill test) also shows significant increases of almost 5 times over the control. The mean±SEM is plotted. N=5 for each group, *: p<0.05

Two functional tests were performed in control (saline-injected) and MP-2-treated (daily intraperitoneal injections of 1 mg/Kg MP-2 for 12 days) mice. As observed in FIG. 6, in the weight test (measure of strength) the improvement with the treatment with MP-2 was 64.4% and in the treadmill test (measure of resistance) the improvement in the animals treated with MP-2 was 25%. The MP-2 treatment significantly improves both strength and endurance.

### Example 9

### High Extracellular ATP Contents Induce the Expression of Proinflammatory Genes

qPCR in Real Time

Total RNA from the isolated muscle fiber culture was extracted using TRIzol (Invitrogen) according to the manufacturer's protocol. The cDNA was obtained by reverse transcription from 1 μg of total RNA, using the enzyme SuperScript II (Invitrogen) according to the manufacturer's protocol. Real-time qPCR assays were performed using a thermocycler (Stratagene Mx300P, Agilent) according to the following protocol. The reaction mix will be given by 1 μL 10×dNTPs, 0.5 μL sense primer (10 pmol/μL), 0.5 μL antisense primer (10 pmol/μL), 4 μL EVA Green (Invitrogen) and 1 μL cDNA. The final volume of the mix was brought to 20 μL by adding nuclease-free water. All primers have an optimal amplification efficiency (between 90-110%). In any case, the analysis of the data was carried out considering a correction of the amplification cycle of each gene according to the amplification efficiency of the primers. The thermocycling conditions were the following: 1 cycle of 12 min at 95° C., followed by 40 cycles of 15 at 95° C. 20 at 60-65° C. 20 at 72° C. Expression values were normalized for the 18S gene and reported as log 2 fold change (ddCt). The Ct value was determined by the MXPro software when the fluorescence level is 25% higher than the basal fluorescence. PCR products were verified by visual inspection of the melting curves.

Increases in proinflammatory markers have been reported in the muscles of older adults, as part of the process of losing muscle mass. Therefore, the effect of chronic exposure to high levels of extracellular ATP on mRNA levels of proinflammatory genes was evaluated. It was observed that when the muscle fibers of normal mice are exposed to high levels of extracellular ATP (300 μM for 2 h), the mRNA levels of proinflammatory genes, such as TNF-α and IL-6, increase. Mean±SEM is plotted, *: p<0.05. (n=4).

Concluding from the above results, use of MP-2 has clear advantages:
It responds to the need for treatment of muscle weakness in older adults, going beyond palliative approaches.
Proof of concept carried out in a mouse model shows significant improvements in muscle function.
Mechanism of action of MP-2 is upstream of many of the abnormalities found in sarcopenia: activation of pro-inflammatory and pro-apoptotic genes, activation of the production of reactive oxygen species.
Low or no side effects are expected from this molecule, due to its low vasoconstrictor effect. This allows us to hope with optimism that this treatment is safe and easy to administer in the general population.

The compounds provided in the present invention for the treatment of sarcopenia differ radically from the currently existing compounds. Currently the most popular therapies are based on hormone replacement treatments, which presents various harmful side effects and with very limited effectiveness. In light of the above background, the DHP derivative MP-2 claimed in the present invention constitutes a new alternative able to ameliorate symptoms of muscle disorders by acting over the biological targets involved in its concomitant loss of muscle mass, showing potential in preventing progression of said symptoms caused by aging.

The present invention offers a simple solution, with a drug with few or no side effects that targets the inhibition of altered molecular mechanisms in diseased muscle fibers that are responsible for the loss of motor function observed in people with increasing age.

### REFERENCES

Altamirano F, Valladares D, Henriquez-Olguin C, Casas M, López J R, Allen, P D and Jaimovich, E. (2013) Nifedipine Treatment Reduces Resting Calcium Concentration, Oxidative and Apoptotic Gene Expression, and Improves Muscle Function in Dystrophic mdx Mice. PLoS ONE 8 (12): e81222.
Araya, R, Liberona, J L, Cardenas, J C, Riveros, N, Estrada, M, Powell, J A Carrasco, M.A. and Jaimovich, E. (2003) Dihydropyridine receptors as voltage sensors for a depolarization-evoked, IP3R-mediated, slow calcium signal in skeletal muscle cells. J. Gen Physiol 121:3-16.
Arias-Calder6n M, Almarza G, Diaz-Vegas A, Contreras-Ferrat A, Valladares D, Casas M, Toledo H, Jaimovich E and Buvinic S (2016) Characterization of a multiprotein complex involved in excitation-transcription coupling of skeletal muscle Skeletal Muscle 6:15 DOI 10.1186/s13395-016-0087-5.
Bustamante M, Fernandez-Verdejo R, Jaimovich E, Buvinic S. (2014) Electrical stimulation induces interleukin-6 in skeletal muscle through extracellular ATP, by activating Ca2+ signals and an interleukin-6 autocrine loop. Am J Physiol Endocrinol Metab.306:E8269-8282.
Buvinic S, Almarza G, Bustamante M, Casas M, Lopez J, Riquelme M, Saez J C, Huidobro-Toro J P, and Jaimovich E. (2009) ATP released by electrical stimuli elicits calcium transients and gene expression in skeletal muscle, J. Biol. Chem. 284: 34490-34505.
Casas, M. et al. (2010) IP(3)-dependent, post-tetanic calcium transients induced by electrostimulation of adult skeletal muscle fibers. The Journal of general physiology 136, 455-467, doi:10.1 085/jgp.200910397.
Casas M, Buvinic S and Jaimovich E (2014) ATP signaling in skeletal muscle: from fiber plasticity to regulation of metabolism. Exercise and Sport Sciences Reviews 42:110-116.
Chow L S (2018) Mitochondria in Muscle and Exercise Diabetes and Exercise Springer International Publishing A G 2018 125 J. E. B. Reusch et al. (eds.), Diabetes and Exercise, Contemporary Diabetes pp 125-136.
Del Campo A, Contreras-Hernandez I, Castro-Sepúlveda M, Campos C A, Figueroa R, Tevy M F, Eisner V, Casas M, Jaimovich E. (2018) Muscle function decline and mitochondrial changes in middle age precede sarcopenia in mice. Aging (Albany N Y). January 4. doi: 10.18632/aging.101358. PMID: 29302020.
Diaz-Vegas A, Campos C, Contreras-Ferrat A, Casas M, Buvinic S, Jaimovich E, Espinosa A. (2015) ROS production via P2Y1-PKC-NOX2 is triggered by extracellular ATP after electrical stimulation of skeletal muscle cells. PLoS ONE, 10(6):e0129882.
Diaz-Vegas A R, Cordova A, Valladares D, Llanos P, Hidalgo C, Gherardi G, De Stefani D, Mammucari C, Rizzuto R, Contreras-Ferrat A, Jaimovich E (2018) Mitochondrial Calcium Increase Induced by RyR1 and IP3R Channel Activation After Membrane Depolarization Regulates Skeletal Muscle Metabolism. Front Physiol. June 25; 9:791. doi: 10.3389/fphys.2018.00791.
DiFranco, M., Neco, P., Capote, J., Meera, P. & Vergara, J. L. (2006) Quantitative evaluation of mammalian skeletal muscle as a heterologous protein expression system. Protein Expr Purif 47, 281-288.
Eltit J M, Garcia A A, Hidalgo J, Liberona J L, Chiong M, Lavandero S, Maldonado E, Jaimovich E. (2006) Membrane electrical activity elicits IP3-dependent slow Ca 2 signals through a Gbeta gamma/phosphatidylinositol 3-kinase gamma pathway skeletal myotubes. J Biol Chem. 281:12143-54.
Espinosa A, Henriquez-Olguin C, Jaimovich E. (2016) Reactive oxygen species and calcium signals in skeletal muscle: A crosstalk involved in both normal signaling and disease. Cell Calcium. March 3. 60(3):172-179.
Fielding, R. A. et al. (2011) Sarcopenia: an undiagnosed condition in older adults. Current consensus definition: prevalence, etiology, and consequences. International working group on sarcopenia. Journal of the American Medical Directors Association 12, 249-256, doi:1 0.101 6/j.jamda.2011.01.003.
Henriquez M, Fonseca M, Perez-Zoghbi J F. (2018) Purinergic receptor stimulation induces calcium oscillations and smooth muscle contraction in small pulmonary veins. The Journal of Physiology 596:2491-2506.
Henriquez-Olguin C, Altamirano F, Valladares D, Lopez J R, Allen P D; Jaimovich E (2015) Altered ROS production, N F-kappaB activation and Interleukin 6 gene expression induced by electrical stimulation in dystrophic mdx skeletal muscle cells Biochem Biophys Acta—Molecular Basis of Disease 1852(7):1410-1419.
Henriquez-Olguin C, Baghersad Renani L, Arab-Ceschia L, Raun S H, Bhatia A, Li Z, Knudsen J R, Holmdahl R, Jensen T E (2019) Adaptations to high-intensity interval training in skeletal muscle require NADPH oxidase 2. Redox Biol. June; 24: 101188.
Jaque-Fernindez F, Jorquera G, Troc-Gajardo J, Pietri-Rouxel F, Gentil C, Buvinic S, Allard B, Jaimovich E, Jacquemond V, Casas M (2021) Pannexin-1 and CaV1.1 show reciprocal interaction during excitation-contraction and excitation-transcription coupling in skeletal muscle. Journal of General Physiology 153 (12), in press.
Jorquera G, Altamirano F, Contreras-Ferrat A, Almarza G, Buvinic S, Jacquemond V, Jaimovich E, Casas M (2013) Cav1.1 controls frequency-dependent events regulating adult skeletal muscle plasticity. Journal of Cell Science, 126:1189-98.
Jorquera, G., Meneses-Valdes, R., Rosales-Soto, G. Valladares-Ide D. Fields C. Silva-Monasterio M. Plains P. Cruz G. Jaimovich E. Houses M. (2021) High extracellular ATP levels released through pannexin-1 channels mediate inflammation and insulin resistance in skeletal muscle fibers of diet-induced obese mice. Diabetes. https://doi.org/10.1007/s00125-021-05418-2.
Kalinkovich A, Livshits G (2017) Sarcopenic obesity or obese sarcopenia: A cross talk between age-associated adipose tissue and skeletal muscle inflammation as a main mechanism of the pathogenesis. Ageing Research Reviews 35: 200-221.
Kawamura H1, Kawamura T y cols. (2012). Extracellular ATP-stimulated macrophages produce macrophage inflammatory protein2 which is important for neutrophil migration. Immunology. 136(4):448-58.
Nair, K. S. Aging muscles. The American journal of clinical nutrition 81, 953-963 (2005).
Potes Y et al. (2019) Overweight in the Elderly Induces a Switch in Energy Metabolism that Undermines Muscle Integrity. Aging Dis. 10(2):217-230.
Romanick, M., Thompson, L. V. & Brown-Borg, H. M. (2013) Murine models of atrophy, cachexia, and sarcopenia in skeletal muscle. Biochimica et biophysica acta1832, 1410-1420, doi:1 0.1016/j.bbadis.2013.03.011.
Scicchitano B M, Pelosi L, Sica G, Musaro A (2004) The physiopathologic role of oxidative stress in skeletal muscle. Mechanisms of Ageing and Development 170:34-44.
Soriguer, F. et al. (2014) Modifications of the homeostasis model assessment of insulin resistance index with age. Acta diabetologica, doi:10.1007/s00592-013-0523-5.
Srikanthan, P., Hevener, A. L. & Karlamangla, A. S. (2010) Sarcopenia exacerbates obesity-associated insulin resistance and dysglycemia: findings from the National Health and Nutrition Examination Survey III. PloS one5, e10805, doi:10.1371/journal.pone.0010805.
Theurey P. and Pizzo, P (2018) The Aging Mitochondria. Genes 9(1), 22. https://doi.org/10.3390/genes9010022.
Valladares D, Almarza G, Contreras-Ferrat A, Pavez M, Buvinic S, Jaimovich E, Casas M (2013) Electrical Stimuli Are Anti-Apoptotic in Skeletal Muscle Via Extracellular ATP. Alteration if this Signal In Mdx Mice is a Likely Cause of Dystrophy. PLoS ONE 8(11): e75340.

1. A compound having the chemical formula: 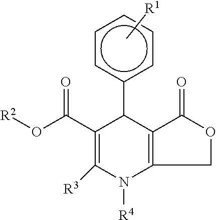
wherein:
R¹ is nitro;
R² is hydrogen or alkyl C₁-C₃;
R³ is hydrogen or alkyl C₁-C₃;
R⁴ is hydrogen, or
a pharmaceutically acceptable salt thereof.

2. The compound of claim 1, wherein said compound is selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridin-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridine-3-carboxylate;
propyl 1,2-propyl-4-(6-nitrophenyl)-5-oxo-1,4,5,7-tetrahydrofuro [3,4-b]pyridine-3-carboxylate;
propyl 1,2-propyl-4-(3-nitrophenyl)-5-oxo-1,4,5,7-tetrahydrofuro [3,4-b]pyridine-3-carboxylate;

3. A pharmaceutical composition comprising a compound having the chemical formula: 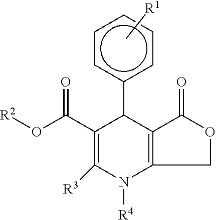
wherein:
R¹ is nitro;
R² is hydrogen or alkyl C₁-C₃;
R³ is hydrogen or alkyl C₁-C₃;
R⁴ is hydrogen, or
a pharmaceutically acceptable salt thereof;
wherein said pharmaceutical composition further comprises at least one pharmaceutically acceptable carrier.

4. The pharmaceutical composition of claim 3, wherein said compound is selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridin-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridine-3-carboxylate;
propyl 1,2-propyl-4-(6-nitrophenyl)-5-oxo-1,4,5,7-tetrahydrofuro [3,4-b]pyridine-3-carboxylate;
propyl 1,2-propyl-4-(3-nitrophenyl)-5-oxo-1,4,5,7-tetrahydrofuro [3,4-b]pyridine-3-carboxylate.

5. A pharmaceutical composition for prevention or treatment of a muscle disorder in a subject in need thereof comprising a compound having the chemical formula: 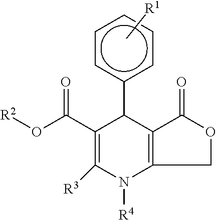
wherein:
R¹ is nitro;
R² is hydrogen or alkyl C₁-C₃;
R³ is hydrogen or alkyl C₁-C₃;
R⁴ is hydrogen, or
a pharmaceutically acceptable salt thereof;
wherein said pharmaceutical composition further comprises at least one pharmaceutically acceptable carrier.

6. The pharmaceutical composition of claim 5, wherein said compound is selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridin-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridine-3-carboxylate;
propyl 1,2-propyl-4-(6-nitrophenyl)-5-oxo-1,4,5,7-tetrahydrofuro [3,4-b]pyridine-3-carboxylate;
propyl 1,2-propyl-4-(3-nitrophenyl)-5-oxo-1,4,5,7-tetrahydrofuro [3,4-b]pyridine-3-carboxylate.

7. The pharmaceutical composition of claims 5 or 6, wherein said muscle disorder is selected from the group consisting of cachexia, sarcopenia or muscular dystrophy.

8. The pharmaceutical composition of claims 5 or 6, wherein said composition is administered via intravenous, intramuscular, transmucosal, intrathecal, systemic, intraperitoneal, ophthalmologic and/or subcutaneous manner to a subject in need thereof.

9. The pharmaceutical composition of claims 5 or 6, wherein said subject is a mammalian.

10. The pharmaceutical composition of claim 9, wherein said mammalian is selected from human, dog, cat and horse.

11. A method for prevention or treatment of a muscle disorder, comprising administering a therapeutically effective amount of a pharmaceutical composition comprising a compound having the chemical formula: 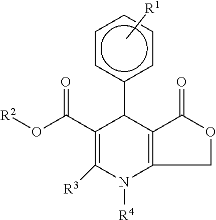
wherein:
R¹ is nitro;
R² is hydrogen or alkyl C₁-C₃;
R³ is hydrogen or alkyl C₁-C₃;
R⁴ is hydrogen, or
a pharmaceutically acceptable salt thereof;
wherein said pharmaceutical composition further comprises at least one pharmaceutically acceptable carrier.

12. The pharmaceutical composition of claim 11, wherein said compound is selected from the group consisting of:
methyl 2-methyl-4-(2-nitrophenyl)-5-oxo-1,4,5,7-tetrahydrofuro [3,4-b]pyridin-3-carboxylate;
ethyl 2-ethyl-4-(2-nitrophenyl)-5-oxo-1,4,5,7-tetrahydrofuro [3,4-b]pyridin-3-carboxylate;
propyl 2-propyl-4-(2-nitrophenyl)-5-oxo-1,4,5,7-tetrahydrofuro [3,4-b]pyridin-3-carboxylate;
methyl 2-methyl-4-(3-nitrophenyl)-5-oxo-1,4,5,7-tetrahydrofuro [3,4-b]pyridin-3-carboxylate;
ethyl 2-ethyl-4-(3-nitrophenyl)-5-oxo-1,4,5,7-tetrahydrofuro [3,4-b]pyridin-3-carboxylate;
propyl 2-propyl-4-(3-nitrophenyl)-5-oxo-1,4,5,7-tetrahydrofuro [3,4-b]pyridin-3-carboxylate;
methyl 2-methyl-4-(4-nitrophenyl)-5-oxo-1,4,5,7-tetrahydrofuro [3,4-b]pyridine-3-carboxylate;
ethyl 2-ethyl-4-(4-nitrophenyl)-5-oxo-1,4,5,7-tetrahydrofuro [3,4-b]pyridin-3-carboxylate;
propyl 2-propyl-4-(4-nitrophenyl)-5-oxo-1,4,5,7-tetrahydrofuro [3,4-b]pyridine-3-carboxylate;
methyl 2-methyl-4-(5-nitrophenyl)-5-oxo-1,4,5,7-tetrahydrofuro [3,4-b]pyridin-3-carboxylate;
ethyl 2-ethyl-4-(5-nitrophenyl)-5-oxo-1,4,5,7-tetrahydrofuro [3,4-b]pyridin-3-carboxylate;
propyl 2-propyl-4-(5-nitrophenyl)-5-oxo-1,4,5,7-tetrahydrofuro [3,4-b]pyridin-3-carboxylate;
methyl 2-methyl-4-(6-nitrophenyl)-5-oxo-1,4,5,7-tetrahydrofuro [3,4-b]pyridin-3-carboxylate;
ethyl 2-ethyl-4-(6-nitrophenyl)-5-oxo-1,4,5,7-tetrahydrofuro [3,4-b]pyridin-3-carboxylate;
propyl 2-propyl-4-(6-nitrophenyl)-5-oxo-1,4,5,7-tetrahydrofuro [3,4-b]pyridine-3-carboxylate;
methyl 1,2-methyl-4-(2-nitrophenyl)-5-oxo-1,4,5,7-tetrahydrofuro [3,4-b]pyridin-3-carboxylate;
ethyl 1,2-ethyl-4-(2-nitrophenyl)-5-oxo-1,4,5,7-tetrahydrofuro [3,4-b]pyridine-3-carboxylate;
propyl 1,2-propyl-4-(2-nitrophenyl)-5-oxo-1,4,5,7-tetrahydrofuro [3,4-b]pyridine-3-carboxylate;
methyl 1,2-methyl-4-(3-nitrophenyl)-5-oxo-1,4,5,7-tetrahydrofuro [3,4-b]pyridin-3-carboxylate;
ethyl 1,2-ethyl-4-(3-nitrophenyl)-5-oxo-1,4,5,7-tetrahydrofuro [3,4-b]pyridin-3-carboxylate;
propyl 1,2-propyl-4-(3-nitrophenyl)-5-oxo-1,4,5,7-tetrahydrofuro [3,4-b]pyridine-3-carboxylate;
methyl 1,2-methyl-4-(4-nitrophenyl)-5-oxo-1,4,5,7-tetrahydrofuro [3,4-b]pyridin-3-carboxylate;
ethyl 1,2-ethyl-4-(4-nitrophenyl)-5-oxo-1,4,5,7-tetrahydrofuro [3,4-b]pyridine-3-carboxylate;
propyl 1,2-propyl-4-(4-nitrophenyl)-5-oxo-1,4,5,7-tetrahydrofuro [3,4-b]pyridine-3-carboxylate;
methyl 1,2-methyl-4-(5-nitrophenyl)-5-oxo-1,4,5,7-tetrahydrofuro [3,4-b]pyridin-3-carboxylate;
ethyl 1,2-ethyl-4-(5-nitrophenyl)-5-oxo-1,4,5,7-tetrahydrofuro [3,4-b]pyridin-3-carboxylate;
propyl 1,2-propyl-4-(5-nitrophenyl)-5-oxo-1,4,5,7-tetrahydrofuro [3,4-b]pyridine-3-carboxylate;
methyl 1,2-methyl-4-(6-nitrophenyl)-5-oxo-1,4,5,7-tetrahydrofuro [3,4-b]pyridin-3-carboxylate;
ethyl 1,2-ethyl-4-(6-nitrophenyl)-5-oxo-1,4,5,7-tetrahydrofuro [3,4-b]pyridine-3-carboxylate;
propyl 1,2-propyl-4-(6-nitrophenyl)-5-oxo-1,4,5,7-tetrahydrofuro [3,4-b]pyridine-3-carboxylate;
propyl 1,2-propyl-4-(3-nitrophenyl)-5-oxo-1,4,5,7-tetrahydrofuro [3,4-b]pyridine-3-carboxylate.

13. The method of claims 11 or 12, wherein said muscle disorder is selected from the group consisting of cachexia, sarcopenia or muscular dystrophy.

14. The method of claim 11 or 12, wherein said composition is administered via intravenous, intramuscular, transmucosal, intrathecal, systemic, intraperitoneal, ophthalmologic and/or subcutaneous manner to a subject in need thereof.

15. The method of claims 11 or 12, wherein said subject is a mammalian.

16. The method of claim 15, wherein said mammalian is selected from human, dog, cat and horse.
